SEQUENCE LISTING (42 sequences)

SEQ ID NO: 1 - DNA, 801 bp, Homo sapiens
  misc_feature (401)..(401): SNP C/A (code M), maps to 4143470133 (rs13102150)
actggtaaacacctcagtatgaaacagtcattttgctcaattgttttgtagatacaatagagcttcactctacagaatccttgaaatgtgagtcattaaagatgacatgtcttcagaataacagaggggttacctatttaagtaccaaatatagtgcatacagatgtgtctgtgtgtgtgtgtgtgtgtgtgtgtgtatacatatatatatattaatttaacgttttgacagaaaatcattctaaaatgtattaaattttataaggcttccttaaaagcacattaaacataatgcaattttctttgatggcccaaagtcaccattgtgaatattaattattatactgtgctataataaaattatgtctgtgaggccctcaaaaatgggtacgctctattgmctctagaattacacaatgtcagaatgaaatgggacctgagagcttgtccagcattcccaattacagatgggacactaacatcaagagaagagtgatgtgatttaattaagcggacacaacagttatctaccccaggtctcccaacttcttatccagagtcctttcacttaacccgcactgcacagcatcatcacctatgctttcatttgttcttctgctgccttttgaagtttttttctcttctccctggttagggtctatgttcacccccatcactgttgttggtgatgaaatctcatcctcccatactgtgcactgtaatgataggtggggtagttctggaatgtgggagggcacgggagcccatgaccatcagacggtaatgactagatgtcagttt

SEQ ID NO: 2 - DNA, 801 bp, Homo sapiens
  misc_feature (401)..(401): SNP C/T (code Y), maps to 4143509994 (rs2874870)
acaaataaggaagtgtggcactggtgcaagatacaaggttaatatacaaaagtcaatttctttgctatacattagcaatgaacaacttgaatttcatgttaaacattttataattttataatatataaattttataatttttaagttatataattattaattccaaaaaatgaaataagaataaatataacaaaatatgtatgaggtctatatgcagaaaactataaaaatctgatgaaataaatccaagaagttctaaataaatgaaaagatatcccatgctcatagattgaaagactcaatattatcaagatgttaattcttctcaaataggtctatagattaaacaaaaccccaaaaatctcagcaattaattttgtagatatcagcaacagcaaccygattgtaaattttttatggaagagtcaaagaaatagaatagtcaacgcaatactgaaaaagatgaacaaagttataggactcatactatctgattttaagacttattacaaaactacagttatcaacattgtgttgtaatggcaaaagagtagaaaaaaaatggaagaatgaaacaaatagaaaacccagaactagacccacacaaatatagccagttgatttttttccaaagaaacagacaattcagtagggaaaagatagtcttttcaacaatgtgtcagaacaaccggacatccatatacataaaaataaccaagcacaaggcttccataacaaaaagaaacatagacctaaatgtagaatataaaagctatactcgatgtccttttatttggtggt

SEQ ID NO: 3 - DNA, 801 bp, Homo sapiens
  misc_feature (401)..(401): SNP G/A (code R), maps to 4143230028 (rs2636638)
ctatacatttactttcgagctggtttcttgttttcttcacctcagtataaataaaagaaggattctctcaccctcaaagttagaaaataaatcttttatctatgtgaatactcactgaacacattgtaaaagaatgaatgagaagagtcttctctatagcacatgatgctaatgtaaaagaataatttaatataaagctattatatctttgctatcactgtcattataattatataatttaagataatttatttaattttttaatattttaaatataattcaagggacagtgttttctgaatatcctttggtgctgtttctatcaggggaaaataaagacaaaaaagggaaagcttcactagagttggagctcagaggtctgaagtgtggctctaaatcarttattctgtccaaaaatgaaaatctggacaattacattacttcttccggcccccaggctcaccatctactgctaagcactgtcacttcttatttccctgtcttctctcttatgaagtctgcggtcttgaacaagttgctccactactctaggccttagttgctgaaaaaaatgcctatgataacttatctttctcatagttattgctatgagaaaattaaagaaatccagacaaagaaagcatctagccaggaaaataaaacatctcccataaatagaactttgcatagtgattaagatcatataaccagaagtcagatgccttaaaagtatctaatgtgcagcttaaacacttttgggaaaaggcaaagtattttttaaaaagtctatgagaaatgcct

SEQ ID NO: 4 - DNA, 801 bp, Homo sapiens
  misc_feature (401)..(401): SNP A/G (code R), maps to 4143500223 (rs4975311)
ctttcaacccttatctccaaatatagtcatcttctaaggtactgggggttagggatttaatatatgaatggggggacatatttcagcccttaacaaatgctgtcctttagaaataatacaacacgaatgagcttacaattggacctcactttactgctacaactttttaaagtgatttaaccatcttgactgtgatctattacattttcatgtaaattttagtctcagagagaaattatagtcctcttaaatttaagttcccagggaatctgttactgtggtctcttgtaactaaaaagtgaatatgttttattgcctatttctaccaaaaaagacaaacacacatacaaaccataaccacaattttgctaaacctaacatgttaaatattgtattgaacrtctgcgttcatttatttaatagaatagagttcctaaaacaaagtctacatgttctctactttcatacagccaacatttaaatgggggaagtagggagggaagcaagtgttaaataggtaaacaaataaatgcatatctcatttgtaatgtgagttataaaaagtgataaatgctgtgaaggagataatgtcagtggattaaggggagaagaaacttgttttatacagagtggtcaaagatggcctttcagagttagaaatatctcattcaaagcataagaaagagacaagtgtgcaaagaaggaaagaagagtggttccgaatcaaactatgatagttaattttatgtgtcaacttgactaagccatggagtgcccagatatttagttaaatattattct

SEQ ID NO: 5 - DNA, 801 bp, Homo sapiens
  misc_feature (401)..(401): SNP C/G (code S), maps to 4143481466 (rs16998560)
ttttatacactttaaaaatctacgataagaatagagctaatgccattctgtccctctcacttgcctagaggcatctaagtggtatatgaggactggtatctctaaaagcctcatcacatctcaggacatcacatcagataagcagcaggaaataaacataaacaaatagttcctctccaatttcacagaagggggtttatcaagacatgttttctctattgagattgctatggaagtgaggcgctatagtgagatctaatcctcaatgctaacagcctgcgctttccttccccgcctcagccatgtgcctctcctaagcatgctgagccggaaaagggtcagcctagacatggccctcttttctaaagctgtgacactgcatgggcacaagacaatcaggstcctctgtggaatttaagatacttcttaattctgaaagctcacaaatactactactactgtattagtcagagttctctagagggacagaactaataggatagatgtacatatgaaggggagtttattaggagaattgactcacacgatcacaaggtgaagttccacaataggctgtctgcaaactgaggagcaaggaagccagtccaagttccaaaacctcaaaagtagggaggccaacagtgtagccttcaatctgtggtcgaaggctggggggccctggcaaaccactggtgtaagtccaagagcccaaaagctgaaaaacatggattctgatgttcgagggcaggaaacatccagcacaggagaaagatgaagctcagaagactcagcaagtctgct

SEQ ID NO: 6 - DNA, 801 bp, Homo sapiens
  misc_feature (401)..(401): SNP C/G (code S), maps to 4142963604 (rs2667092)
catagccatccatcagcaagtcagaccattcagcaagatctccactactgcctgtcatctaccgtcacttgttctcctactgtcagaagctgttttcctgtcttttaccatgcttgcccctctattaagtttcactaactcctaatatatccatttagacacatagcaaagcaaccaatgcacaatgctagtggcatagtaaaaatacttcccgaagagcttccatggaatcaaaattcagctgtatcctagctatgcacagggagccaagcttgtcaaaaaagcagtggtgcttatccaactgcaggaaagtggaaatcttttcatctgctcatcaccaagagattgaagctcttgaatctcaaggaggaagtttatctacagcctctttctgtacaatstcttgtttttaaacttgtttctcaagtttttcaacccccttttccactgccaatttatgtgtgtgtgtatgtatgtatgtatgtatgtatgtatgtatgtatgtatgtatttatttatttatttatttttagacagagtctcactccgtcgcccaggctggagtgcagtgatgcgatctcagctcactgcaacctccgcctcccgggttcaagcgattctcctgcctcagcctcctgagtagctggaattacaggcacgcgccctcatgactggctaatttttatatttttagtagagatgaggttttaccatgttggtcaagctggtcttaaactcctgaccttgtgattcgcccacctcggcctcccaaagtgctgggattacaggcatgagccaccg

SEQ ID NO: 7 - DNA, 801 bp, Homo sapiens
  misc_feature (401)..(401): SNP A/G (code R), maps to 4143444744 (rs13107432)
gcaaatacaaaccccagacctactaaatcagaaactctgactctgacgtgtggaccaacaattcatgttttaactagcccccagataattcgaatgcatactcaggtttgagaatgtgatggagcatgtttggagtatgatgagtgggaggtgatgctgagacacaaggtgaacatgatgtaagaggattatcagaactgaagatgtacacttgtcatgcaaagggctgaaattcttcttttctgatgcctttacttgctttcagttataggttttattttgctgaaaagcttcccaatctcagaataatttctcagctttcaaattctccttgcaatccacctacttggcaaagcaaggccagggttgtaacccttaatcagtctccactcttcccacartgtccctcctgcattgctctcctcctgcttttagcttttcttttttcaataaaagtatgtattattttttcttttattattatactttaagttttagggtacatgtgcacattgtgcaggttagttacatatgtatacatgtgccacgctggtgcgctgcacccactaactcgtcatctagcattaggtatatctcccaatgctatccctcccccctccccccaccccacaacagtccccagagtgtgatgttccccttcctgagtccatgtgatctcattgttcaattcccacctatgagtgagaatatgcggtgtttggttttttgttcttgcgatagtttactgagaatgatgatttccaatttcatccatgtccctacaaaggacatgaactcatc

SEQ ID NO: 8 - DNA, 801 bp, Homo sapiens
  misc_feature (401)..(401): SNP G/C (code S), maps to 4143020338 (rs336307)
gatacaagagtttaaggaagagagatgggctggagatgtagatatgaagattgatagttaatagcaggtatttaaagcaactaggctgtgttcaattggggagcagagtagacatgcatggactgaatcctaggtcactccaacattagatgttggaaagataatgaggaatagagatttaaaagaagcagccatagagcaggagaaatcacagagtgtggtggctgggaagccaagtgacaaaagaagtaagaaataactatgtcacacttgttactagatcaaggaagatgaggacaagtgactcagcattgggtttaacaatgcaggctcttgacagaaagtttccatgaagttacatggataaaagacttactggaatgagtttaggtgaaaaaatsggtggtgaaaaattttaattccacttttatctactctttttgggacattttcaaacttaagcttgtcctgcagtataacataatattcctctctattgccaatcccttttacacatttttatgtgtctatgcaatgacaaatatgaacaatatatgaagaattcctcagccatctcgatgtttccaaatatacatatgttttaaataaaatatgcaaaatatttggataactgactggatttttcatttatatatttggatgtctacatggtatgacagttgagatgtttataaagcaaagttattggaagtaaattttgtttattactatgtaatagtctaatgtatagtttctgctggaatgacattcatgtgttaatttaagaaaatctttgtatct

SEQ ID NO: 9 - DNA, 801 bp, Homo sapiens
  misc_feature (401)..(401): SNP C/T (code Y), maps to 4143295246 (rs1353624)
aagagtttgttcctgtggtaaggatgaagacaagtacatatttaaaatataaatatagaacattttaatataaataaagagtcatttaggaaatattatctttggctatattcccagttgagagctttcctgtggttattgttaaagcaaacaaacaaaaagcaacttttccaaagttaattatagtactggaatataaataaaaatcatgtaataatattaagaacagcagcaatataaactgtcgtgcattgagcagccacggtatgacaggcactctaccagaagcttaaggagtcagtaattagaccatataaaacaggtggcaaaaaaaaggaagagaaaaggagtgtgaatgaacatgcagaaagattgttttagtttagggtgtgtttgaacaygtttgtagataaaatgggacaacaattatctcattttgtccttacaaccaccctacgtaatacacacgcttattattccccattttattgataagtaaagtaaggctctcagtgcttaagtaactggcccatgattacagaattaattgtgttgaagtctggatataaaccctgattcaggtgagccacagttcacattcttaaccacatagaggccattgttctaagcaaagatgcattttgtatgctataatttttttcttataacttttctttcagtcatctagatttctcagcaaagcttacccagataggtaaacattttggaagatgtagaatagagaagattttttagaaggggagaagtgggagagagctttccatattgcaaatttttgag

SEQ ID NO: 10 - DNA, 601 bp, Homo sapiens
  misc_feature (301)..(301): SNP T/C (code Y), maps to 4143081071 (rs2636637)
tagatttccctttgtgtgtgtgtgtaatatgcatatactttggagaattcttgaataatttatatgtatcgattcagtaataacaccctgttcctctgccatggtacaccttcacgtttctcccatcgtattcggcccaactagaatttctctcaccatatttatttcctttgtttattcatgtttcctagctattttttcgtatcattttatgaggccattcttctgtaaactttagagcacctagagcagcatcttacattatagaaccttgagcgagagggatttgcgagatatctaygtctattgtcgtttaaatgtgaatacttttaaaaagtacttccagaaccaaaacactggattgcaacaatgaaaatacttgttgcatctaagtagtagtaacatggagggttttttattctttttctaaaaaataactttcatgttttatatagtttggcaataaataaggaaacataagagtcttgtaactgtggcatctttacttggatgacagattctcacttaaacatataattcattggctagcaaataaattttatcaggatttcaacttccaaaaaaaaattgcccctaagga

SEQ ID NO: 11 - DNA, 801 bp, Homo sapiens
  misc_feature (401)..(401): SNP T/A (code W), maps to 4143269745 (rs1391098)
ggtagatccctaccctaaccctgctactacaacaaggtcattgaagagcctgtctccacaagcctctaaatcaatcctttcattattgattggcttggcaggagataaaaatgcagtgacctttgccattttgagggagtaaggcatccaccattcagtcactttactatttggtcttgtttaaggaaaatatttttttccagaatattttaccttaaacagagaagtcattaaacctgactcaaatcaggttaattactgagttccagcaccagcttctccctcctgcccctccaattctccccagatatagatggtgaagagacactaaaaatcatcagtatgtgggaggccccttctctaacctcttttcttcatccacacaattgacaagtgcaaawccagcctataaggaaataaaagaacttctttctcaataagagcttggtgacacattaatctaaagcaacattgctccaaggctgcaaactagaacaggctcaactcagagcaatgattgtctaaaatgtccctagtgaacaatttgaaggaagagtccttccctctatagccacagaaggttatcagagcaaaaccttatctgtcataagactccccagatatcaagttcagtgagtgcctaatcccaggagctgttctgctggagggaaaagtgatttcaggcacttcgcagccagctcagtgcagggggctggaggatgatgttatgggaagctggtatttacagctcagccttgccagcaccaatgaggaagagtgttggagcagatgttagttttc

SEQ ID NO: 12 - DNA, 601 bp, Homo sapiens
  misc_feature (301)..(301): SNP G/A (code R), maps to 4142976133 (rs2667101)
ttgctaagactagtgctatgcttataggagactggatagtcacaaaagattcctatatcatgcttaaatacatccactgacagctaatggcattgttgggcatcgagtatttgggagatggagataacaatacacataagggacagttaatctttattatgtactgtctgtaagagaagaatagatgaaagtctctatatctgttaacttattcagaatgatgtgttccactcagcttgcgtggctgctttaaaatcagtcaatttcaactataatcattcactaaagattgcctactaaragataagagtggccaggtatgtacatgtgtatatgtttgtgtgtgcatgtgtgtatgttagactccacttacttactagtttgaaacttcaagattcctgatgttaagtcactattttaataatttttttgtggacctactttgtgcacttataattccaaagtaatactaactatggaactgattggcatggagaaaacaaccaagaacacgcttttcaatagtgtgatacattaaagtttgatggttaaataaaatactattaatggtttattaattaataatgataatcactgattt

SEQ ID NO: 13 - DNA, 554 bp, Homo sapiens
  misc_feature (54)..(54): SNP C/T (code Y), maps to 4143459907 (rs2059510)
tattttgaatataagtagtgggtatttacatgttcatattgcaggcatggatgygaaggttaaggaaagtaaaaatatttttcccaaaattgttttaacaaaacatgtgtcctattgaaataagacttccctcaacttataattatgaaccatgcattttcttgccaatgcggtaaaaaagagagaatccatttaaaaaaagaaagagagagattaaaattagtgacaatggtgaggctggaaattttatcaattatacctcaaaaaagataaaagtaaacctcaaatatttgtacaagacaatgaaaaaaattgtttagacttgatcctggcccttttcaggcctttttttgtgattatatataattacaaaattgttattttatatatatatatataaacaatttaggattttatttccctctaagtagcaaatttttttctttgagataataaaatatatatatatatatactgccagatacctgagtgtctgagaaagtcaaaaacagtcagatggcatttattttgcatatcaacatgacaatataaca

SEQ ID NO: 14 - DNA, 801 bp, Homo sapiens
  misc_feature (401)..(401): SNP G/T (code K), maps to 4142969773 (rs2667096)
ctcttaacaactgtgctatacttttttcctacattctccattgcacgagaaactgcagattatagtacttgcacacatggccttacagaatatgtaggacagctcaacactcagcataattggagagtaataggtagtattctctgtaaatgaaatatatgttctgaacctaaacagttatcagagtgggcttaggcttagggtgacttgtgatgtccccagttcatacctattgtcccagaaggatttacatggaaggatggaagaagggcatctggtgaaagatgacaacattaaaaaagagagaaaaagaaataaagagaggcaagggtttgttatgtttgtgtgttacatgtttgctatatgagttggagggggcaggtatttatgtgaactaaggktgaacgataagtgggggagagcaacagagaggtgtgctctgataaagggatgagcagatgaggtgggcaaccagcagaggcaaaagcagcttgagagctggttaaagaaaagctattgcaagatcatagtattatacatgatatacttggatttaaaatattagcttgaaaatactgtgcatggtagattgaagcagagcagttggaagcagaaaagccagtgagattattgcaaagaatattgaaataatcaaggtgtgaggcaaataaaataggaccagggtggcagaacatatcaatgccaaaggacacatcgagggggaagaaatggcagaattttgggcaacagactagtaattaagacttacatcattgtttacaattacattgtcaacaactt

SEQ ID NO: 15 - DNA, 801 bp, Homo sapiens
  misc_feature (401)..(401): SNP T/C (code Y), maps to 4143014855 (rs336296)
catggacatcatggagcagagagaaaccatccccagtttccctttctgatacctaacccacagaatctgtgagcataaacaaaatgattctgattttataacagtaaacttgaggtagcttgttatctggcaataagcaaccagaacagaacagctacaataaaaacagtttaaagagcttgtacttctaagtatgaaaaagttaacatgatgaacactgctttcactgcctactctggtgagtactatagtctgtaacatcaagtctcttctacaattaattctaaaataattatagttaactgtactagcaagcagacaaatgcaattaggtaaaagccattgagatatcttaaaactgaaaattatgttgttttagatactgtaaaatcttgaaaaayaagaacagacaaaactccaccaaaaccagctttatttaaattgcatttccaattttcaatttatgaagaaggcaatacaaaaaaatcaaatgtcaagatcccatcctggtctgcgttgaatatttttgacatacacactttcagtgccttgtcttaaacaaatttcaaggttgcatcacccatagcaaaaggaaaaaaacaaactttaaaagtaatttttaaattatatccaaagcaatatattaaaaattcttcaaaatttcctcaatcaacattatacattggagaccatggaatatggtgttcagggagaagaatacgacgataacatatttttttcctgtttatatttttttaaagacagcattcataatttgatttcagaatttccaaacaattt

SEQ ID NO: 16 - DNA, 701 bp, Homo sapiens
  misc_feature (501)..(501): SNP T/C (code Y), maps to 4143084950 (rs1219275)
tgcctcagattgtctgggatataaatcgcccagtgtatccgtgctgtatatgctactagcccactagtcacttaggtgccatctgggtcaccagatcgactgtcaggttattgcagtgctcatgttcaagtagccttgatgtggaagcctaacactacatcattcccctcactttatctaatcacgtagacattgtaccatctaacatcatcacaagaagacacgtgagtacagtacaagaagatattttgagagagaccacatttacataactttcattatagtatattgttataattgttttgattattagctcatgttagtctcttactgtgcctaaatttaaattaaattgtatcatagctatgcatgtgtaggaaaaaaacagtatatacagggcttggtactatcaggggtttcaggcatcccctgggcgtcttggaacatatcctcgtggataagggggaactattgtattgctgttcttgcaactgggacgayactaggaaagttgaagtcatggaaaggatgtgaccatatcactgagttgcctactgactgttctaaaaaacctttttcttttctcatttagcgtgaactacattaaatgaaaaaagaaagtctatctaaatgttatcataagagtagggcaaacaatttatttaaaatatcagcttaataatatcgtgtaaaaagaataa

SEQ ID NO: 17 - DNA, 801 bp, Homo sapiens
  misc_feature (401)..(401): SNP A/T (code W), maps to 4143341540 (rs1391092)
gcttcagtgaccaatgctctcagtaattagacaatggagggagaaagtaattaggtaaaccacaaagaagtagaaataaactgtatgggatatgttggtcatggatgaaagacaaattaaaaaaaagtataaaggaaacatctaggaaaaaataactaataaataatttgcttaatccatgttgaataatcaatttaagcctgtgaagttaaacctgattaaagatatcccttaattaaaaagttcctacaatgtattacttagaaagaaacatgacaagaagaatagatctattttatgtcaacaacagttcagttaaatgatatagtcctggcaaggagaagtgggagagaaaagtttgaggtcttatatacttctcatttattaattgatgtatcatwtctcatatctgcggggtgcaccaaatgtgaatttaaaccagtgtcactaaaaacaggctacaatccaactgcagaacatgctaaatcttggtcaccagaaaaccactacccacccgacagatgtgtaaagaagctctaattggttcattttctcttccttcaattaaataactatatcagaagtacaaaaacctcatgtaggccttagataaccttttattgctgttctctttcccattcctgtacttttaatcatagaccacaaagaacatggcctttggaattaaaatccagattgctctacattctagatataggccttcacacaagtgaatgaacttgtctgtgaaacataggtaatgggaggcaactgttgagcctgtaataggaacaaaggaga

SEQ ID NO: 18 - DNA, 635 bp, Homo sapiens
  misc_feature (246)..(246): SNP G/A (code R), maps to 4142916958 (rs1995960)
cagtaaatttgtgttataggtggaaggagaccagaccacgttttctacagatggaaaatcctgaggtcaagatgagtagaagtgagatagttatacatactgatttttcttcccaaatccattactcaaatatatcacttttcccctttggggtaagttgggggtagagacaaggaatggatatccttctagacccacagaaaaatagagaaaccctcctcccttcacagcaacattcctcagccrttgtcgtatttgcccattcagttcaatcaataaatccatagtcaacctgtaggaagaaattagcaatctgtgagtttctgttttcactttcctgaggctgttgtccttaaaatcaccaaagttcctttgagaatactactaatatcactgtattgccaccacaaatcccttaaatgaggcagaatagaataccaataactccaagcccctgtgtttcattctcgttttgttttttaacaattagactcagcagccttgtccaaaatagtatattaaagtttctgtaataagaataaatgataatgtaaaggttagcctatataaaatgacaacaaatgaagatgcccaaatttatttccattattaaattctagaggatacttataacc

SEQ ID NO: 19 - DNA, 801 bp, Homo sapiens
  misc_feature (401)..(401): SNP C/T (code Y), maps to 4143229643 (rs1907106)
acgttggcttgtccatctcctagctgagcccagtgtctggaacagagggctgcactgtgagtgcatacagtgtgccaagaacaatgcccatgagtcaggtttatccattataactgtcagccacttgcttgatctcaaaggataatgaaaatggtcacaaataataaaatctgttaaaaatcaatctggataacgatggcatcataccattacatttcgtaaacttgtcagacaagaggagttattatttgggtaccaatgctatcctttttttaatcctaaaaataactgtatatcaggactgcatgttgtataaactacattaataaaatcaaaaaatgtgaggacaattgttttatgctgcaacaacaaacaatgggattgcctatacatttactttygagctggtttcttgttttcttcacctcagtataaataaaagaaggattctctcaccctcaaagttagaaaataaatcttttatctatgtgaatactcactgaacacattgtaaaagaatgaatgagaagagtcttctctatagcacatgatgctaatgtaaaagaataatttaatataaagctattatatctttgctatcactgtcattataattatataatttaagataatttatttaattttttaatattttaaatataattcaagggacagtgttttctgaatatcctttggtgctgtttctatcaggggaaaataaagacaaaaaagggaaagcttcactagagttggagctcagaggtctgaagtgtggctctaaatcagttattctgtccaaaa

SEQ ID NO: 20 - DNA, 801 bp, Homo sapiens
  misc_feature (401)..(401): SNP C/A (code M), maps to 4143304592 (rs1497389)
gcctcttagcaatgagttttctcctttagttattaacaaaatggattgtgttgatagatgtactgatattaagccacttttgctttcctgaattaaacattacttggtccgagtataatttttttcatagattaatcctactgaacactgttacatttataatctttgcacctatgcttttaattgaaatttacttatagctgttaaaaaaacattcatcaggttttagtattaaacctacctagtcctcataaaatatactttggaagctttctttttttcccaatatagtcacagtcatactgcttatgtaccagtgtttcaaatatttctattacaatacccataaaaggtgcttgaaagaataactgcttaccacctaggctatgaggttgtgggtmaaacctaactgcctaaatttctacttccttatcatgagcttcctaacatcttctgagcagacacccatcaaaacagagttatgtggacatgtcaaaattccaatctgtggacacaataggctcaaagtagatctgccttgcaacttaactcaaccactcctctcaatagtatcccatgatgcttaaaaatgaaactgctcactattcaaacactgataagagaaaaaaaagtcatatttttaccagaaaaaaaaatctattgtttttggacatgtgtagtaatagttcagaaatataagcttcaaggataatcattaatattcacaacagtggttgcacaggcaggagggaaaggagaggagattgagaagggggatatagaggaccttgactttattta

SEQ ID NO: 21 - DNA, 801 bp, Homo sapiens
  misc_feature (401)..(401): SNP T/C (code Y), maps to 4143343632 (rs12499068)
tctgtccagtaatgataatatgggagagatattatagtatgaactcagactcctgggttcaaatcttaggctcactactttaccagtttaattctaagttttcttacatagaaatcagggatgataaaagagcaattactcataatgaaatgataagaagtaattaatatatgtaaattatttagaacagcatatggcaaacaagtaagcaatcaataaatgttagttacttttaatattattatcctgtagagttgtggtgagaaattcatgacatgctgtttgtaaagttaagcccataatagatgctgaaaaaatggtagttgttgctgtgtcctcctagggctttgaacaatgacttgtcattagaaggcgcccagaaaatggtagttcagggaaaytcaatgttttataagggatgtgttacactataagaagcaagaaggctaggtgtggtggctcatgcctgtactcccagcactttggaaggctgaggtggtagattcactggagcccaagagttcaagaccaacctgggcaacatagtgagaaccccctctctacaaaaaataaaaatgaaaaaaactagccaggcatggtcatgtgcacctgtggtcctagctacttgggaggctgctggtggaaggattgcttcagcctaggagttggaggctgcagtgagctatgattatgccactgtgctctagtctgggtgaccgagtgaaaccttatctaaaaagaaacaaacaaacaaaaaacaaaaaacagaatcaagaaaatggatgtcaatcaacttataat

SEQ ID NO: 22 - DNA, 801 bp, Homo sapiens
  misc_feature (401)..(401): SNP G/T (code K), maps to 4142920229 (rs6821787)
aaaataaatcgaacaattttcaaaagccaaagcagtttatgaccttaaagcatttagcaaacctaatatctgacctgcataatctagacaaaatgtattttatcaataatctttaaagctgttttttgagacagagtttcactcttgttgcccaggctggagtgcaatggcatgatgttggctcactgcaacctctacctctagggttcacgtgattctcctgcttcagcctcccaagtagctgggattataggtgcctgccattgacgctcagctcacctcagcctcccaagtggtgggattacaggcgtgagccactgtgcccggcttctaatgatttttttgtgcagaaatacatagtatcacaagagtggagacatggattttaaaattctgagctktaagaaacttactcctcagataaacacatgcaaatgagtccatgcatttatataactgggagctctctgaattaacatttatgtggtatatacaatttattaaagcataaccatgtttgagacattcacatatgtatttacatgtaacactagtttattttttattgtacaaatttcattgaatataccaagttttattctactattgatggacttttgtgttatttactattttgaaatattatgaacgataaaaaatcagaccatcttttattcttttttctgttttagttgttttgttcaactctctaaatgcaaggaacactctaatttccttcacccagggcttatttcttccaccactccattctaggtatgccactgcctaggagtagaattg

SEQ ID NO: 23 - DNA, 801 bp, Homo sapiens
  misc_feature (401)..(401): SNP C/T (code Y), maps to 4143243556 (rs2635429)
tatgattcagccactattataagcactggcaatctcagagagtaagagattaagaagacaaggtatatttcgttgatacgcaataacaatggcatggtaagagtcaaacatctaataataattgctaagaatcttcagctttctaaaatctatcagctagtcttagataacggttttctagttgtttaattttaatcttgcatggagaaaatgatgcatgtacaaacctaaatgatttaggtcacagaatcttagagatgcaaggaatctctcagttaatctaaaaaaaaaactaggcaatatgaaaattatatctgaaatattcctgggaagtacattgaacatttcttcttcaacatttggaatttctagcttactatctcacaaatcagtatcataayggtagtgttacaaactcacatgcccacaggctccaggtaggtaaaattaatgaaaggagtggcctgggtgtttgagacaataggaattggagaggactctggcaaaatggaatcacatttctagtctataagaagtgacaactacccagcttcaacggattgaaaaaattctgagaaatattccttcaaatattaacaatgggtaaatattaattcttttgaaagtgggctattattaccatatgctgatttttaccagaaacatgctttgtaactaaggtgagtgggccaaagcaagcccactactattttagtaaaattagtttttaaaatgtgctaccctaaaacagttgttattaaccttgacaacacagtagaatcgcctgtaacatgtttttgg

SEQ ID NO: 24 - DNA, 801 bp, Homo sapiens
  misc_feature (401)..(401): SNP T/A (code W), maps to 4143294023 (rs1391095)
tacacattctatgcctgtatcaaaatatcacatgtaccccataaataagtacaatcattatatgcatagaggaaggtaaaaataaaagcagaaaatatcatatgcattgggttctacaatggaaaagacagagagggttgggaagcatttcaatgatgggcacatctctcttcccaaggtcttatttcatttccagtacgacttctgacagctggtagaaatgtgtgcctctgacagagcagtttcacacattctcaagtgcaaaaagtaagattgacggcatcctttccatttccatcatcagctgaagtgtctcctctctaggaaggagtaattagctcagtcttctagtactggaagtcaaactaaagcatcttgactacactgactcctgctgcctwcctagtctcacaaataggaattcatatgattaagcacattttacttcaatctaggccttgaaccaatgacctaccttctataactgcctacacaatacatacatacatacacacacaggggcatgtagtgcacttctcttctggacttgaaatacagatggttttaaactgaatcatttatattctatgccagaatagaaatcagtctcaaatagctgcaggctctaacaatgactattttgaaaatccaactccatattatgcagcatagtggaaagagctctgattgaagagccaagtcctgtatgtgtcataaattctatttttgcatctttattgaatagattggtgccctttcataaagcagaaagtgtttgagatgaatagagtaacaacattt

SEQ ID NO: 25 - DNA, 801 bp, Homo sapiens
  misc_feature (401)..(401): SNP G/A (code R), maps to 4143364802 (rs1817970)
tctttatggacctcacccaaatgcccatgagaatgattggaagcagagtgggtgcctggagaaagtgtccctttgaggtacatgcctgctgaaggggaacagcagtaattgcaggaaaagtacaaagacccacttggatcctctttcctacagaacaaaaatgacagaccctgctgaccttaggtgaagactcattgaaattagaaagggaggagcagtaaatcttcctgggatgacaccactagagatcctctgatcctttattactatagagtgatcggatcactgagaaagtcccaccccaagacccaagaacatcgggcctgtctacaaccaagcatgaaagatgacgatagagaatgcctctgcccccgccaccaggctgagacaccaagaaacargtgacagaaggctacctcccaccagagggggcatgcaagcacaaataatgaacctctctcaggtagaaacttccaagaaagcttaaagtagagagtggagagtggagagcagtctccaggcatctaaaccgcactctaagcacaatttaatattaaagtagaataaagaggggaggaagaagagaagaagataaaggagagggagaaagaagaggaagagaaggaaaagtggggcagaagaggagaaggatgaaaaagtgtcaaaagcaaaagcaaaaaaataatatgtacatatataccaagagacccagactcagatattacccaggtattagaactaacagaaagggaatatgctaaaaactctagtggaaaaagtggagaagttgcatgaccagat

SEQ ID NO: 26 - DNA, 801 bp, Homo sapiens
  misc_feature (401)..(401): SNP A/C (code M), maps to 4 143453079 (rs17717651)
tttgtgtcgtctatgagaaataatttctcttttctgagtctatgttgatgatgtagctttcgacattattataacatgatttattatataatccacactttagtaaaatgacagttattctctgaatggaataaaatttcttttgagtagattttttttttaacaagcctgcgtgaaggtcatatttgctgtcaattccacagtctccgactaaggggaaagaccctaccaaatatagtaaaatacaagtcagtggtagactatttctcaacaaaggagccagaatttgttaatgacatttacaaaggccagttcttagttgcttctcaaaatactaattttaaaatattgactaagctgaatataactgataaatataaacattattgatttactttttmatatccaatttcaccatgttaattttattttctaattattattaagacatattgacttgtcctgttctttgccaccaccatcctggttcagataatcatctgtagcccaggctgctgcaatatttttcaaattgatttctgcattcacttttatcctgctctgctttttctcaacaaagtagtaggaaggatcctcttagaaacgcaaatcaggtaacatttccctccgcttaaacactttcaaaggattccagttgttcctaagataatgaaaaaaccacttgagggcctgcatatgcctgcagttggcctctccatccacttcagcagcagcaccccctgctctcattattcttcaccttggcctttccaggaatagccctgctccctcccagtcgaa

SEQ ID NO: 27 - DNA, 801 bp, Homo sapiens
  misc_feature (401)..(401): SNP A/T (code W), maps to 4142844783 (rs967003)
tgtgcctgtgtctgtggtgagaacacttaagatctgctctcttagcaaatttcaagtctacaacacaatattattaactgtatattattattaattatactccatactatacattacctctccataacttacatcctgaataactgagactttgtattctttgaaccacatctcccctttggctccatccctatcccctggcaatcaccattctattctctgcttttaggagtaacgttttaagattccctgtaaaaacgaggcaatgaagtatttgcctttctgtgcctgaattatttcacttagcataatgtcctccaagtttatccatgttgttgaaaatgtcaggatttccttctttttaaaggctgaatagtattccattgcatatatatacgccwtattttctttcttcatctgttgatagataccgaagtagtttgcgtattttggctattgtgaatagtggaatgcacatgggcgtgcagatatctcttcaagataatgactttattttctttgaatatactccagaagcagtgtttctagtcaatatggtggccatatttttaattttttgaggatcttccatgttgtttttaataatagctgtactagtttacatttctaccaatagtatgtgtgagcattcccttttcttcacatcacgatgtttatttattcattgctaattactacatgcaagcagagtataacaaatgtcaataataagctataaacttctgaatattctaaagttcttaccatctgagatcagttttataactcccttatgcttaa

SEQ ID NO: 28 - DNA, 801 bp, Homo sapiens
  misc_feature (401)..(401): SNP G/C (code S), maps to 4143163452 (rs2627798)
acactttcttgcttccagtacacgatagaagcagcatatttaaatgaatccaacttgattggcttcaggcagtacattcattattttactacactgcagattcttgtagagaatctattcattccatagttttaattatgtagttgtttaattaattattcagctaaatgtatctatattgcctattctccaaaagctacattatactttctaaggataagtctgtaatccaattagacactgacacatcctcatttgtatttgaagatatcttaaatacataataaatgaaacttggctcttttaaagatcggatgagcactgagaaatctaaacagaaattttacaaaaacactatccttagcatggcttcagaaaagtgagggtaagaaaaacatgasaggagctcagaaaatacagcaaacagataccctgaatgcagccaaacccacaagctcctggccaaaaataagaaaaactattgcagatgtcataataaaacacaaacgtattatttgtctgcagttggtgacaattggtgaatttcatctgtagaatttggatttgagatcttgggtggttagaaaaaggattggccttagttctgttcattctgtttcagtgagtgaaggatacaacttgtaaagctagaagctagcttctatttcatgctgaaaaaaccaagacaacccatactctattccactcatcaagttgtttcttgccagtatgcctctgatcaaggcattccatgcaaatcacaggaaccccccactttcctaaccacatgtaatcatccta

SEQ ID NO: 29 - DNA, 801 bp, Homo sapiens
  misc_feature (401)..(401): SNP G/T (code K), maps to 4143232882 (rs1872292)
gcttgaactttagttctgaaatcttacctgccattttgagttctgatgggtcacaaaatttgttagattttctactgtattattacagtaatttctagaatcccccacccatttatttctggtggttctaataatacataaaatggttggcaaaataaaatcatggtgtgctttttgatttattcaatacatagttcatagatttgctagcttccaagcacaaagccaggaatattagaaatagagactttgctctcatggagtacatctagcaggagtgtggagacatgactgattgcctatctaactcactttctgccctttcttcattactaatagcacctcagttttgtttaaatgagaattgtacccagattctcttctagctaaggacttaggckctccccaatactatgtaagtgacagtatacaaaatgaaatttcccaggaaattattgttttccagaattgaagggataggccagctgagctttttgcccttgatttttcactcaacccagaatgtgctacctggggtaccatggccagtttgtgcaattctctaagaaagaaggaccaggaagatggatagagtctggttgcttcaagtcacttttgagtagctgaagccctggacaattttcccaagacttctataacatgagaaaaacaaacccattcattagttttaagccagtttgtcacgttacttgccttagggtacgttcctaattgcaaaaataggcatgtcacaggataccaattgagatttctcttttagtccaggaattatagccgagt

SEQ ID NO: 30 - DNA, 701 bp, Homo sapiens
  misc_feature (201)..(201): SNP G/T (code K), maps to 4142999991 (rs168059)
atttatatacctttgctcatcgtaaataatttgaaactgctacagagataagcacaatacaatagaaaatacttaatatagataaggacaaaataaaactaaataaagagtgatatgataaccttggcgaactttatttttcccttgagtttttatgatgaagaaaccttggtgaatttttaagaaatccagagaaaatakcctggaaggtgattggagtaattcaaaggtaaaataaactgcaactgatgttaaataaaattaaaagttgcagtaaattcaaaaacatacaaatcaaattcctttaaaacactgatttttaaaaatccagagttgttccccagaatgtctgtaaagttcttaaaaaattaaatcctgggacccacctcagatcttctcaattataatccatgtgaggtctaaacatatgtattgcgttttagtataggacatgttgttttattataaaattgaacccaagatgatgataacttaaactacatcttgatacatttttaaaaatagatttaaggggtacaggtgcatctttgttacatggacatactgcatagtgctgaagtctgggctttcagtgtaaacgtcacctgaatagtgtacattatacccaacaggtagtttctcatccctccctcccttctcatccttccaccttttaacacaccaatcaaacatatgtattt

SEQ ID NO: 31 - DNA, 801 bp, Homo sapiens
  misc_feature (401)..(401): SNP A/G (code R), maps to 4143234593 (rs2636670)
ggcttcctccccactgcacgttgctagagtcacagaaagtctagcccgtagggggaaatggtgggaggaggtagaagaaaataaaatcagaaaagagactcaaggtttaaaataattaagatggattcccaagaacatgagtgagagaaaaaaattggagaaatctacacgactgggccaaaatatcattaaagcttcaagcaagggggattcaacacaagagcagtggtggagtggggtcggggcaggcagtgaacacaggagaaaaaaattaaaccagctaagcttccttgaaccttgataaataaatggaacaccttgatagaataatgtattccccatattaatactttaaaaagtctcacattttatgaagcagtgccaggggtgtgatgagagtrtgcatttaggatggagggtccagttggaagcagggccccagggtggtactagtggaaacatgtgagctgagccctgagctgtggaagggagccctctccccatgaagtgcagcagcccaggctgacagaggtccatctgcctgtgtctgtggaggtctgaagctggggagaactttctggatgcactggaggaagtaaatgaaggtttgtgtagtcagcacagagattggtagtagaagtgaatggcacaagaggttgaaggcagaaaaactgcaaggactccctcaagtcaaagcagttactcaactagtcccaaggaagagatgagaggcttgtgttaggcatgagaaagcaagacatcgcccttttccctctgctttttcactaacagctgcagaga

SEQ ID NO: 32 - DNA, 801 bp, Homo sapiens
  misc_feature (401)..(401): SNP T/C (code Y), maps to 4143344473 (rs3756125)
ttaattcttagccgttggttctcttattgcatcctatgtggtgaagtaagagttttatcaatagaataaatgcaaaaatatacctctgtgcttggtattcccttcagctagcatgccatgtaaaaggcaaaaatactaaagaagatatcctaaaaccctccattctcccactggagttttaaaaaagtcagagctctataatcatttatagaacacagaccagcaagagagtaggattgtgaacatagaagataattcataagttgggcctcttatgatttttccttttagaaaaacatataaaacaatgcctgatttatagcaattcataaagtatgaatggacctgacattcaggttaatgaaggcccttgcaagttgtaaatgctgtgtctgtaaagygatgagacattttcttgtgcagattgtcagttggggcacatttctatacatttttaaatgccgagcacagaaaactgatcacatgattttatcttttttttcccctaaatgggctactaaaatacagtacctgcatatataaaatccaaaatcagttcacattatttaatacatgacctaacagacaaagtatttagaatcagctctctcctggacaaagccttaaccaccaaagaggataagcactccttatttatagccacgataatatttgaattaacctacctaaggacctcttgtcattgtaccagtacatattgttgtgaaaaaactgaattttaatcattaaactctaggacggtacacatcatttacctggtacagatgtatactgaattgc

SEQ ID NO: 33 - DNA, 634 bp, Homo sapiens
  misc_feature (204)..(204): SNP T/A (code W), maps to 4143130219 (rs1994217)
cactttgagtaagtagagtaattgcttccacacctgtttccttgctaagttctgtcagaatgtcttggtacatattcaccatttgatcacagtgagtaaggacattttttcgcagattgtcccaatgtggagaaagctcaccaagttcttttatctcctggtttctgttaagagtggaagagaagagaaactttatttttatcwtaaatccataaacgcataacccttagtcagagttgtcaagataataagaaattatttcatcctaattcccaggaaagttctatgagcttattagcttaaatattttttaaaaattattttagcacaaaaaaatatgaatagcttaaccacacaaataaaaacattatgtcaaagggcaaaaaattagaaatgttaaattccctgataaatttttcacaaagatggcagacttcctattcttatatacatagggaagacatttagttaaaaagcaaattgtaaaacctctatgtaaagatggctaagtttttaagtttcaactgtacaaaatcaaagaatgtggaaagggctacctgaccatgttggaagcaaacatgagatacttagtcaattcagtttatatataatatagttacaaatatttttttaa

SEQ ID NO: 34 - DNA, 801 bp, Homo sapiens
  misc_feature (401)..(401): SNP C/T (code Y), maps to 4143277467 (rs2029990)
tcttttcttaacctcaagagaaatgaacagaattcaataagattacctatttttaaaatgccacatactccaatagatactactggggctacaaaaaattagaactatgtagttttatcctccccaaatctttcatattgataagggcaaaaaataggatgacacaattggtttacaatgtataaaatgataaagtgctggacagaaggtacaaattacaatacaagttcaaaagagaaagattatattagttaatctagaaaatgtacaaaggaggtgatattggcagaatacctttaaagatggggtaaaacttttgccaggggagaaaggcattctagataaaagggagagaggagagcagtgagtgttgatttgaaaataagaaagggtaattcaaytgtgtaagaatgagaatgtacagagatgtgtggcctacttttctcagagacaggggtcaagtaagggagaaatctaaatcttatataaatgtttgaaatgatatgagaaggaaatgtgaggaaagcttggatagctttccatccagaactcaagggttggggatcctggttagcaggagagcaaagaccaggacctgcaggagtgatgaggagctgggaggataaaaggttgctcttgagttagtaaggtttcagttgtgaagtagtacaaataacatatttggtttctgctcttagttcctggcacagaaccctaaaacctttgtaatttcctgagtgatagaagtgttaggtgcatcttttgtccttttatttggtctttggcctcagttcctgacat

SEQ ID NO: 35 - DNA, 937 bp, Homo sapiens
  misc_feature (437)..(437): SNP C/T (code Y), maps to 4143053786 (rs168061)
gcctgaaacaactcaacattttgttaaaagggtgctagtgaatatgtttgtagagaataagattggagaaggtttttttttttcttggagagagcagaggcaatgcatcatagactctaagagggcaggctctggagccagaccaccagggttagaacccaggctgtgcacaggtcactacaactctctacttcttcatcctttaattttgtagaattaattctacagtgttgtaagaattaaatgagttaacgcagtcaatatttttttttaccacatatcaagtaatccataaatatgagcttctatgttaaaacaattcaaataaacaaagtaatttctacatgatactttgcactcgccctcctaccagcattgattctttatgctcttccttctgtcctatttctgaggatgaactgttgtctagctcagaycatctcctctgttggtacaccagatctcatcctctctcacctacttaagagtgtcactctagcaattctctcccttcctcccacattatcaatatttcctttttaactggatcattcttgtcagaaaacaaacatgctattatctcctcatcttgaaacaaatatcactgttttgaccctaattccctttacagctcctatactttatttctccatttcctttcacaatgaaagttctcaaaagagttgtctatacaggttgtctctacttcctttacttcctctttcttttaacccactctacttaattcctacccactctagtaaggctactctcgtcatcaaagacatccgtactgctaaatccaaagccatctcttagtcctcattttatttgacctattttctaaccccggctttcaggatagcatattctctttgtattcctcctacctcactggccgtctcttgcccaactttgttgctggttccttcactta

SEQ ID NO: 36 - DNA, 801 bp, Homo sapiens
  misc_feature (401)..(401): SNP C/T (code Y), maps to 4142833334 (rs1500847)
gtatgtcaagcaagggacccagaggtgccagtgtgcctattgagagggcagagtggtgcttgaatctaggtgaagtgaccatggcgggcaaggatgtagaaaccagtggcaaatggccagaattgaggactccagtgaagggtgccagcatgtcaagcttaaattgctaaaggggactcaatcatagtgagaagtcacccgtcctgggaatcaatagcccagctactccgcagtgaagacaagaggatcctgagaggtcaggtcaatctgaggaccccagcctctccagtgctctatgatcataggaaatggagatggtagagagcacagaaaggaaaatggcctgtgaggatcagaggatttgcaggttaaataaggggcttgcaatctcttcttatacygtaacatgagcaaatttctggcctaactccaggtgttgcctatctgtatatttctaaaaactgaagcattctcctggaaagtgaatcactgagggtgtggagtccgggatagaccctttaggattatgttctcacttcttttctcatctatcccttcttttttacatatctcttgttcaagaagcaaagacctttccattctgtttgccgtatcttcttgtgctatggccataagcctcttggattcgttctcttttcagaggggagattctctccaaagtctattaaataaacactttttctacatcctccaggtatacctctctaactgggagctgtggtatgacatggaagtagaaacctctgaggatgaaaattatttgctattctatcttctaaa

SEQ ID NO: 37 - DNA, 801 bp, Homo sapiens
  misc_feature (401)..(401): SNP G/A (code R), maps to 4143333792 (rs3775707)
catacattaaaagatatatagagcacttaaaaagcctaatagcccctctaattactttattagtataatatgcttttctatttttaatctcctttgcctttttttgttttttattcttccttcccctcaagaattttaagacagcaattttctgaagcctaggatgtcaacctctccagctcagatctcctgagtacattcagaagaggatgcttcctaggctcagtgcactacaatccatgataattcaattatatatagtaaaatgacagaatgtacagtgtccctgcctatactaatatccacacaatgagaattgcacaagcagttggaaatactctcttcactactatctgtttatggttatttcttagaagaatatgtctctatatcatgctgarttgatttatggccatgaatatatatcctcccatctttcttctgtattctctgtaaaatctttgagtctttaattcattgagagtagctctcatcttgtccttcctctcattttagcttattttcaatcgggcaagcctgctttttataattttcctttgacattattcttttttcttctcttctctgccatctcatatttcctctccaacatatgtgttactctcatcactctgaaagacgaggttatcaaagtcaccactcatatattttataagacttgggactctaggagtcaagcacacactgctcttaacaaaccacagcttctcccctcagttcactggttttaaagtctttatagaaacaaaacaaaaatgtgttatgttctccttaactttt

SEQ ID NO: 38 - DNA, 601 bp, Homo sapiens
  misc_feature (301)..(301): SNP C/T (code Y), maps to 4143216769 (rs2636645)
gccacaggtctgtcaatgactgtggatgggggtatagcacctgcatgtggatggaggcatgatgtgtggggaattggggggtgagtgttcatttgggtgaggatcacatgcctgcctatggatgtgtgtgttacagaatatgcttatggcagaaaatacacatagacaaccactcacacatttctttgaaaaatacatttttgttgttcttctaaatcagcattttctgaataacatgactatgtttcttggaacatagtgtctaataagctgttgataagtattccatgatgaaagcttyttgcattcaaattaatttaagaaaagctgcaggcgacacgtgtcctctgagcatcttcgagggccttgaaaggctactgaaggctctgagatgtcctacaggaaagaaattcactttcccttttttttttcttttcttttttttttagaggactttcaaacttatttggccacagaattcctttattcaaaaaacatgtattaataaccaaagggataccggttttacaaaacaacactggaaaaagcagatataaatgtgtccagtgaagcagaaacacagtgataataatgaaactga

SEQ ID NO: 39 - DNA, 801 bp, Homo sapiens
  misc_feature (401)..(401): SNP T/C (code Y), maps to 4143224971 (rs3775692)
ataaaagtcagaaataatcagtgcaaaggaatattttaaaatacaacacaaaacaaaaatgcttcttaaggctgacctaaggctcttctaactcaatacttaagagacagaatcagtggaaagaaaataaattacatcagaatttttaaaaaagccatctaccagaaatcaagaggctacgataaatctataatctgtctggttcatagggagagaaaaccacaaatttggcaaagcacaaaactgggaaaaataataacctagccatttcagaattggttttctaacattattttttctagaaaagtcaattcttcgttgttgaatttgttttatataatcaagatcaaatgactaggaatcaggggtattaaaatcaaaagtgaagaaatttgtcttgytttcatctaaatatgaaatttatgtatttcttttctctttcaagggtaataccctaaacttgacccttcggttcttgtcctctctctaaggtactgtgtgttcactaattttaattttactaagaactgcttgtcccctgatttctgtttcaaaccaatgtctaaatgttgcactagtgtgtagtatgctcctgatgttaatgactgttatgttataatgaatactggggcttgtgagtcagtaagatgaagaagaacatgctatgcatctgatctagaaaacatgtggactgctagtagtcctaaggcaactcaatgtttgacatctcatgttatgaatccaggggcatatgggtcacattcaatagtcaggctagaatgatgatattttctaggttat

SEQ ID NO: 40 - DNA, 801 bp, Homo sapiens
  misc_feature (401)..(401): SNP-1 invention A/G/T (code D), maps to 4143470133, inventive variation 1 (rs13102150)
actggtaaacacctcagtatgaaacagtcattttgctcaattgttttgtagatacaatagagcttcactctacagaatccttgaaatgtgagtcattaaagatgacatgtcttcagaataacagaggggttacctatttaagtaccaaatatagtgcatacagatgtgtctgtgtgtgtgtgtgtgtgtgtgtgtgtatacatatatatatattaatttaacgttttgacagaaaatcattctaaaatgtattaaattttataaggcttccttaaaagcacattaaacataatgcaattttctttgatggcccaaagtcaccattgtgaatattaattattatactgtgctataataaaattatgtctgtgaggccctcaaaaatgggtacgctctattgdctctagaattacacaatgtcagaatgaaatgggacctgagagcttgtccagcattcccaattacagatgggacactaacatcaagagaagagtgatgtgatttaattaagcggacacaacagttatctaccccaggtctcccaacttcttatccagagtcctttcacttaacccgcactgcacagcatcatcacctatgctttcatttgttcttctgctgccttttgaagtttttttctcttctccctggttagggtctatgttcacccccatcactgttgttggtgatgaaatctcatcctcccatactgtgcactgtaatgataggtggggtagttctggaatgtgggagggcacgggagcccatgaccatcagacggtaatgactagatgtcagttt

SEQ ID NO: 41 - DNA, 554 bp, Homo sapiens
  misc_feature (54)..(54): SNP-2 invention A/G/C (code V), maps to 4143459907, inventive variation 2 (rs2059510)
tattttgaatataagtagtgggtatttacatgttcatattgcaggcatggatgvgaaggttaaggaaagtaaaaatatttttcccaaaattgttttaacaaaacatgtgtcctattgaaataagacttccctcaacttataattatgaaccatgcattttcttgccaatgcggtaaaaaagagagaatccatttaaaaaaagaaagagagagattaaaattagtgacaatggtgaggctggaaattttatcaattatacctcaaaaaagataaaagtaaacctcaaatatttgtacaagacaatgaaaaaaattgtttagacttgatcctggcccttttcaggcctttttttgtgattatatataattacaaaattgttattttatatatatatatataaacaatttaggattttatttccctctaagtagcaaatttttttctttgagataataaaatatatatatatatatactgccagatacctgagtgtctgagaaagtcaaaaacagtcagatggcatttattttgcatatcaacatgacaatataaca

SEQ ID NO: 42 - DNA, 801 bp, Homo sapiens
  misc_feature (401)..(401): SNP-3 invention A/G/T (code D), maps to 4143453079, inventive variation 3 (rs17717651)
tttgtgtcgtctatgagaaataatttctcttttctgagtctatgttgatgatgtagctttcgacattattataacatgatttattatataatccacactttagtaaaatgacagttattctctgaatggaataaaatttcttttgagtagattttttttttaacaagcctgcgtgaaggtcatatttgctgtcaattccacagtctccgactaaggggaaagaccctaccaaatatagtaaaatacaagtcagtggtagactatttctcaacaaaggagccagaatttgttaatgacatttacaaaggccagttcttagttgcttctcaaaatactaattttaaaatattgactaagctgaatataactgataaatataaacattattgatttactttttdatatccaatttcaccatgttaattttattttctaattattattaagacatattgacttgtcctgttctttgccaccaccatcctggttcagataatcatctgtagcccaggctgctgcaatatttttcaaattgatttctgcattcacttttatcctgctctgctttttctcaacaaagtagtaggaaggatcctcttagaaacgcaaatcaggtaacatttccctccgcttaaacactttcaaaggattccagttgttcctaagataatgaaaaaaccacttgagggcctgcatatgcctgcagttggcctctccatccacttcagcagcagcaccccctgctctcattattcttcaccttggcctttccaggaatagccctgctccctcccagtcgaa

(19) United States
(12) Patent Application Publication
(10) Pub. No.: US 2013/0078628 A1
(43) Pub. Date: Mar. 28, 2013

(54) Single nucleotide polymorphisms of human chromosome 4 in the inositol polyphosphate-4-phosphatase type II gene (INPP4b gene) for the diagnosis or pre-diagnosis of multiple sclerosis

(71) Applicant: Saleh Ibrahim, Lubeck (DE)

(72) Inventor: Saleh Ibrahim, Lubeck (DE)

(21) Appl. No.: 13/580,642

(22) PCT Filed: Feb. 21, 2011
(86) PCT No.: PCT/EP11/52500

§ 371 Date: Dec. 13, 2012

(30) Foreign Application Priority Data
Feb. 22, 2010 (DE) 10 2010 008 827.7

(51) Int. Cl. C12Q 1/68

(52) U.S. Cl. CPC C12Q 1/6883
USPC 435/6.11

(57) ABSTRACT

The invention relates to a single nucleotide polymorphism (SNP) of the nucleobase at base position 143470133 (rs13102150) of human chromosome 4 in the inositol polyphosphate 4-phosphatase type II gene (INPP4b gene) for the diagnosis or pre-diagnosis of multiple sclerosis or for determining the risk of contracting multiple sclerosis.

The invention relates to a single nucleotide polymorphism (SNP) of the nucleobase at base position 143470133 (rs13102150) of human chromosome 4 in the inositol polyphosphate 4-phosphatase type II gene (INPP4b gene) for the diagnosis or pre-diagnosis of multiple sclerosis or for determining the risk of contracting multiple sclerosis.

Diseases which cause degeneration of the nerve tissue cover a wide range of different disorders in young and older people. Such diseases result in a decrease in functionally active neurons in the brain and/or bone marrow. Neurons can die off for various reasons: depositing of proteins or protein fragments, metabolic failure, toxins, inflammations, blood circulation disorders or head injuries. All these factors bring about a far-reaching loss of neurons and glia cells, for example in the event of a stroke. In other cases they cause a loss of specific groups of neurons and functional systems, in Parkinson's disease (PD) or amyotrophic lateral sclerosis (ALS) for instance.

Nevertheless, the clinical features and/or characteristics of the individual diseases are often almost indistinguishable. This causes difficulty when attempting to make a clear diagnosis. Furthermore, the course of different diseases varies greatly. Thus, in the case of some rare diseases the precise disease can often only be determined as part of an autopsy.

In the young population multiple sclerosis (MS) is the most frequently occurring of the neurodegenerative diseases. Multiple sclerosis is a chronic-inflammatory autoimmune disease of the central nervous system (CNS). It is characterised by the destruction of the myelin sheaths which surround the axons of the nerve cells. In the early stages of MS the patients often recover fully from the symptoms. However, the structural damage remains and with every further recurrence the probability of clinical damage increases. In most cases the damage is restricted to focal areas of the white matter. The structural changes give rise to various clinical symptoms, for example vision disorders, walking problems, ataxia, paraesthesia, muscle weakness and paresis, as well as speech and coordination problems, and also psychiatric difficulties. The cause of MS is still unknown.

In young adults the disease often already begins between the ages of 20 and 30 years, with considerably more women being affected. MS is an autoimmune disease which is structurally characterised by infiltration of peripheral inflammatory cells, which are usually T and B-lymphocytes. These cells are found in the white matter at local destruction points where they attack myelin sheaths. Destruction of the myelin affects the isolation of the axons. In the healthy state action potentials are transmitted by saltatory conduction at the Ranvier nodes. These nodes exhibit no or only slight myelination between two oligodendrocytes on one axon. Damaged isolation not only disrupts saltatory conduction, but due to the change in the ion concentration it also leads to metabolic problems.

With regard to the cause of MS there are only a few conjectures that a genetic predisposition or environmental influences could play a role in the course of the disease. An involvement of biological factors is also being discussed.

Particularly a genetic indicator for MS would be desirable as in this way a clear diagnosis would be possible. The approaches to date show that an increased risk of developing MS is associated with the presence of the human leukocyte antigen (HLA) DR2. HLA-DR2 is one of the definitive genetic indicators for MS in the HLA region. In addition to the HLA-DR2 haplotype other loci also modulate the susceptibility to MS in the HLA region, for example HLA-DR3. However, genomic studies show that further genetic factors also contribute to a susceptibility to MS. Endeavours are therefore being made to find other genetic factors.

The aim of the present invention was to find further genetic indictors which are associated with multiple sclerosis.

In humans this aim was achieved by a single nucleotide polymorphism (SNP) of the nucleobase at base position 143470133 (rs13102150) of human chromosome 4 in the inositol polyphosphate-4-phosphatase type II gene (INPP4b gene) which serves or is used to diagnose or pre-diagnose multiple sclerosis or to determine the risk of developing multiple sclerosis.

The search for genes which are involved in a polygenic disease such as MS is very complicated and extensive. A combination of mapping strategies was employed, whereby an animal model was initially used. Experimental autoimmune encephalomyelitis (EAE) is an animal model for MS. Thereby the disease can be induced in various species ranging from mice to higher primates. The animal model correlates with the human disease to a large extent, for example in terms of acute, chronic dysfunction with relapses and recovery phases as well as demyelination in the white matter of the CNS. In mice EAE can be induced in two different ways, i.e. by means of active or adaptive immunisation. The active inoculation includes immunisation with autoantigens of myelin proteins or with bone marrow homongenisates. Adaptive immunisation involves the transmission of lymph node cells from previously immunised mice or of stimulated antigen-specific T-cell lines.

For analysis evoked potentials (EPs) are used. An evoked potential is an electric potential which is recorded in a patient after a stimulus up to reaching the effector. Muscle contraction can be cited here as an example. EPs show whether lesions are present. For diagnosis various EPs can be used, for example visual evoked potentials (VEPs) or motor evoked potentials (MEPs). Here, for diagnosis the electrical stimulation of the brain was used to trigger motor evoked potentials (MEP) in the muscles of the extremities. Due to the damage to myelin sheaths temporary dispersal at neuronal conduction takes place which results in modified cortical evoked potentials. Cortical motor evoked potentials (cMEP) provide quantitative data on the physiological status and are therefore particularly suitable for a functional examination.

MS and EAE are complex diseases in which many gene loci are involved which could have an effect on the phenotype (quantitative trait loci, QTL). A QTL is a quantitative trait locus, i.e. a variable point on the genome with influences certain traits. Finding such points is very helpful in the search for genes in relation to diseases in which more than one gene is involved. As the trait is not formed by a single gene, each involved gene modifies the trait.

QTL mapping is used to identify potential genes for various traits. In mice inbred stains are used under controlled environmental conditions for QTL mapping, for example C57BI/6, SJL, FVB and C57BU10.S.

For mapping genetic loci various markers are used, for example restriction fragment length polymorphisms (RLFPs), hypervariable RFLPs, mini-satellites, micro-satellites or single nucleotide polymorphisms (SNPs). QTL EAE 31 proved to be relevant.

As a single QTL can contain hundreds of genes, selecting individual potential genes for further examination was a demanding task. Additional fine mapping was therefore indispensable in order to identify the precise chromosomal locus. Used are the establishment of cogenic lines, recombinant selection and advanced incross lines. The first option for producing strains is the generation of backcross populations. The backcross strain implies the pairing of F1 individuals with one of the parent strains (cf. FIG. 1B). Intercross generations are produced by pairing F1 generation siblings, which results in a mixed F2 population (cf. FIG. 1B). It is possible to analyse a mixed population of two strains for various phenotypes. Software tools are used for mapping the QTL and the chromosomal locus.

The differences between various murine strains can be analysed by means of high-density markers or by the creation of strain distribution markers. In addition, single nucleotide polymorphisms (SNPs) can indicate phylogenetic relationships between inbred strains. Determining which fragment has the same or different ancestors is possible through comparing SNPs of various murine strains in the QTL region. By way of the SNPs strains can be subdivided into haplotype sub-groups. The SNPs help to combine this information with the phenotype data.

Another approach is comparative genomics. Through the genomic comparison of various species (for example rats, mice, humans, cf. FIG. 2) common sequence fragments can be determined. Furthermore, the gene locus, highly preserved regions or the quantity of non-coding DNA can be determined. Highly preserved regions within pathogenic loci in various species can be determined by means of comparative genomics. This speeds up the isolation of likely pathogenic genes.

For the fine mapping of the QTL (EAE 31) haplotype analysis, intergenomic analysis and gene expression profiling were used, whereby the inositol polyphosphate 4-phosphatase type II gene (INPP4b gene) was determined as the best potential gene of chromosome 8 of mice (cf. FIG. 3).

Inpp4b (protein) is an Mg²⁺-independent phosphatase which catalyses the hydrolysis of the phosphate in the 4 position of phosphatidylinositol-3,4-biphosphate, inositol-1,3,4-triphosphate and inositol-3,4-biphosphate. The murine protein is 96% identical to human and 90% identical to the rat orthologue.

The Inpp4b gene, as the best potential gene, was sequenced in two mouse strains, the resistant strain C57BU10.S and the sensitive strain SJL, in order to find differences in the sequence. The resistant strain withstands EAE inducing, the sensitive strain reacts to inducing with EAE.

The sequence differences were compared to known polymorphisms which lead to amino acid variants in humans. The coding sequence of Inpp4b of SJL and of C57BL/10.S were cloned (Promega) in a pGEM-T easy vector and produced two SNP differences in the cDNA, which resulted in a displacement of amino acids (AA): c1434C/A (AA 478 S/R) and c1655A/C (AA 552 H/P).

In order to find out whether one or both SNPs are decisive for EAE susceptibility, DNA constructs were produced each containing one mutation (either serine->arginine or histidine->proline) or both mutations. Transgenic mice were produced by pronuclei injection. On inducing EAE it turned out that both SNPs are relevant.

The Inpp4b gene, which in mice is localised on chromosome 8 is on chromosome 4 in humans. The gene itself is known, with the sequence being described for example in Anderson et al., “The cDNA cloning and Characterization of Inositol Polyphosphatase 4-Phosphatase Typ II”, J. Biolog. Chem. 1997, Vol. 272, no. 38, pages 23859-23864. The gene is also listed in the ENSEMBL database (chromosome 4: 142,949,186-143,383,906). Three splice variants are known, i.e. alpha, alpha short und beta. In the alpha short variant the exon 4 is missing.

All the following details about the individual SNPs relate to the definition and/or numbering in accordance with the Ensembl database (Ensembl release 56—Sept 2009; Homosapiens version 56.37a (GRCh37)). The Inpp4b gene and the individual bases respectively are read off in relation to the orientation of the codogen strand of chromosome 4.

In an association study MS patients were studied with 39 SNPs coming into consideration as markers. DNA was taken from body samples and the relevant sequence was identified in the area of the Inpp4b gene. The control group of the study included a total of 349 study participants who did not have MS, of whom 210 were women and 152 men. The group of patients with MS included 362 persons, 4 of whom had a clinically isolated syndrome, 8 were primary progressive, 3 progressive relapsing, 244 were in the recovery phase and 90 secondary progressive.

### Tag SNP Selection

By way of the tagger algorithm in Haploview 39 tag SNPs were selected, which cover all usual haplotypes within the INPP4b gene (http://www/broad.mit.edu/mpg/tagger, www.hapmap.org). The algorithm is based on r². The use of a stringent r²limit value (r²>0.8) between the SNPs allows the selected tag SNPs to resolve most existing haplotypes (see Altshuler D, Brooks L D, Chakravarti A, Collins F S, Daly M J & Donnelly P 2005 International HapMap consortium a haplotype map of the human genome, Nature 437, 1299-1320; Barrett J C, Fry B, Maller J & Daly M J 2005 Haploview: analysis and visualization of LD and haplotype maps, Bioinformatics 21, 263-265). SNPs with minor allele frequencies (MAFs) of more than 0.05 were selected.

### SNP Genotyping

Genomic DNA was extracted from peripheral blood leukocytes using the QIAamp DNA Blood Mini Kit (Qiagen, USA). The genotyping of all SNPs took place by means of a 5′-exonuclease assays (TaqMan assays on demand; Applied Biosystems, Inc., [ABI] Foster City, Calif.), whereby the primers provided by the manufacturer were used. The fluorescence signal of the sample was detected in accordance with the manufacturer's instructions (TaqMan Assay for Real-Time PCR, 7500 Real Time PCR System; ABI).

For each study participant the EDSS (Expanded Disability Status Scale), which is a measure of the severity of the disease, was determined. The Cochran-Armitage trend test was used to test for association with susceptibility to the disease and with the EDSS value. The results for all examined SNPs are set out in Table 1.

The term “rsXXXXXXXX” stands for a designation in accordance with the Ensembl database, the base pair on human chromosome 4 affected by the SNP is shown in the second column, the P column shows the obtained potential values. Column A1 sets out the normal base, column A2 the SNP base. rs13102150 [4:143470133 (codogen strand, forward strand)], rs2059510 [4:143459907 (codogen strand, forward strand)] and rs17717651 [4:143453079 (codogen strand, forward strand)] exhibited significance, whereby rs13102150 was particularly relevant (Ensembl database entries of January 11, 2010).

TABLE 1

| SNP | BP | A1 | A2 | P | OR | L95 | U95 |
|---|---|---|---|---|---|---|---|
| rs13102150 | 143470133 | C | A | 8.516E−03 | 0.729 | 0.576 | 0.923 |
| rs2874870 | 143509994 | C | T | 1.189E−02 | 0.722 | 0.56 | 0.932 |
| rs2636638 | 143230028 | A | G | 1.818E−02 | 0.741 | 0.577 | 0.951 |
| rs4975311 | 143500223 | G | A | 5.062E−02 | 0.777 | 0.602 | 1 |
| rs16998560 | 143481466 | G | C | 5.890E−02 | 0.783 | 0.607 | 1.01 |
| rs2667092 | 142963604 | C | G | 7.155E−02 | 0.746 | 0.542 | 1.03 |
| rs13107432 | 143444744 | A | G | 7.558E−02 | 0.811 | 0.644 | 1.02 |
| rs336307 | 143020338 | G | C | 8.171E−02 | 0.808 | 0.635 | 1.03 |
| rs1353624 | 143295246 | T | C | 1.310E−01 | 0.799 | 0.596 | 1.07 |
| rs2636637 | 143081071 | T | C | 1.488E−01 | 1.22 | 0.93 | 1.61 |
| rs1391098 | 143269745 | A | T | 1.875E−01 | 1.17 | 0.925 | 1.49 |
| rs2667101 | 142976133 | G | A | 1.881E−01 | 0.849 | 0.664 | 1.08 |
| rs2059510 | 143459907 | T | C | 1.998E−01 | 1.2 | 0.906 | 1.6 |
| rs2667096 | 142969773 | G | T | 2.138E−01 | 0.857 | 0.671 | 1.09 |
| rs336296 | 143014855 | T | C | 2.206E−01 | 0.849 | 0.654 | 1.1 |
| rs1219275 | 143084950 | T | C | 2.383E−01 | 1.17 | 0.901 | 1.52 |
| rs1391092 | 143341540 | T | A | 2.593E−01 | 0.869 | 0.681 | 1.11 |
| rs1995960 | 142916958 | A | G | 2.618E−01 | 0.877 | 0.698 | 1.1 |
| rs1907106 | 143229643 | C | T | 2.927E−01 | 1.13 | 0.898 | 1.43 |
| rs1497389 | 143304592 | C | A | 3.370E−01 | 1.13 | 0.882 | 1.44 |
| rs12499068 | 143343632 | C | T | 3.494E−01 | 0.894 | 0.706 | 1.13 |
| rs6821787 | 142920229 | G | T | 3.797E−01 | 0.879 | 0.659 | 1.17 |
| rs2635429 | 143243556 | C | T | 3.945E−01 | 1.11 | 0.876 | 1.4 |
| rs1391095 | 143294023 | A | T | 4.270E−01 | 1.11 | 0.861 | 1.43 |
| rs1817970 | 143364802 | G | A | 4.343E−01 | 0.872 | 0.619 | 1.23 |
| rs17717651 | 143453079 | C | A | 4.609E−01 | 1.12 | 0.833 | 1.5 |
| rs967003 | 142844783 | A | T | 5.069E−01 | 1.09 | 0.846 | 1.4 |
| rs2627798 | 143163452 | C | G | 5.102E−01 | 0.921 | 0.722 | 1.18 |
| rs1872292 | 143232882 | G | T | 5.507E−01 | 1.08 | 0.838 | 1.39 |
| rs168059 | 142999991 | T | G | 5.837E−01 | 0.932 | 0.725 | 1.2 |
| rs2636670 | 143234593 | A | G | 6.327E−01 | 1.06 | 0.837 | 1.34 |
| rs3756125 | 143344473 | T | C | 6.820E−01 | 1.05 | 0.824 | 1.34 |
| rs1994217 | 143130219 | T | A | 6.933E−01 | 0.953 | 0.748 | 1.21 |
| rs2029990 | 143277467 | C | T | 7.027E−01 | 1.05 | 0.82 | 1.34 |
| rs168061 | 143053786 | C | T | 7.221E−01 | 0.956 | 0.748 | 1.22 |
| rs1500847 | 142833334 | C | T | 7.994E−01 | 1.03 | 0.817 | 1.3 |
| rs3775707 | 143333792 | A | G | 8.607E−01 | 1.02 | 0.796 | 1.31 |
| rs2636645 | 143216769 | C | T | 8.664E−01 | 0.979 | 0.761 | 1.26 |
| rs3775692 | 143224971 | C | T | 9.781E−01 | 1 | 0.758 | 1.33 |

Accordingly the invention relates to a single nucleotide polymorphism (SNP) of the nucleobase at base position 143470133 (rs13102150) of human chromosome 4 in the inositol polyphosphate-4-phosphatase type II gene (INPP4b gene) for the diagnosis or pre-diagnosis of multiple sclerosis or for determining the risk of developing multiple sclerosis. Cytosine is normally found at this base position 143470133. In the case of an SNP this base is replaced with another base. In the entire text “another base” is taken to mean that the bases are generally the nucleobases adenine (A), guanine (G), cytosine (C) and thymine (T) and that the term “other bases” in each case covers the group of the three remaining bases, i.e. if a cytosine is normally present at base position 143470133 the other bases are adenine, guanine and thymine, one of which is then present instead of cytosine.

Particularly the invention relates to a single nucleotide polymorphism, which is a replacement of the base cytosine with adenine at base position 143470133 (rs13102150) of the human chromosome 4 in the inositol polyphosphate-4-phosphatase type II gene (INPP4b gene) for the diagnosis or pre-diagnosis of multiple sclerosis for determining the risk of developing multiple sclerosis.

In the event that at base position 143470133 another base, particularly adenine, is present instead of cytosine, the patient is diagnosed or pre-diagnosed with MS or is classified as being at increased risk of developing the disease.

The testing for association with EDSS was carried out by means of the Jonckheere-Terpstra test. Only MS patients were tested. As has already been stated above, the “Expanded Disability Status Score” (EDSS) is a performance scale which provides information about the severity of disability in multiple sclerosis patients. The scale starts with 0 and ends with 10, whereby the severity of the disease increases with increasing values. In determining the EDSS the doctor examines the patient's functional systems (FS). The results are set out in Table 2. Shown are the SNPs with p<0.1 from the MS association test or the Jonckhere-Terpstra test for association with EDSS. Here too rs13102150 turned out to be particularly relevant.

TABLE 2

| SNP | pTrend | pJonckTerpstra |
|---|---|---|
| rs1391095 | 4.27E−01 | 3.60E−03 |
| rs2029990 | 7.03E−01 | 5.50E−03 |
| rs3775707 | 8.61E−01 | 6.70E−03 |
| rs3756125 | 6.82E−01 | 1.51E−02 |
| rs1872292 | 5.51E−01 | 1.63E−02 |
| rs1497389 | 3.37E−01 | 6.52E−02 |
| rs2635429 | 3.95E−01 | 6.92E−02 |
| rs2636670 | 6.33E−01 | 6.99E−02 |
| rs1391098 | 1.88E−01 | 8.21E−02 |
| rs2059510 | 2.00E−01 | 8.97E−02 |
| rs2874870 | 1.19E−02 | 9.64E−02 |
| rs2667092 | 7.16E−02 | 1.27E−01 |
| rs4975311 | 5.06E−02 | 2.23E−01 |
| rs13107432 | 7.56E−02 | 2.94E−01 |
| rs2636638 | 1.82E−02 | 3.25E−01 |
| rs13102150 | 8.52E−03 | 4.70E−01 |
| rs336307 | 8.17E−02 | 4.80E−01 |
| rs16998560 | 5.89E−02 | 7.69E−01 |

The haplotype analysis for MS took place by way of a “sliding window approach”, whereby the window size was set at 3. The result is shown in Table 3 and FIG. 4. Of the haplotypes 1 to 6, haplotype 1, which has SNPs of the nucleobases at base position 143470133 (rs13102150), base position 143459907 (rs2059510) and base position 143453079 (rs17717651) of human chromosome 4 in the inositol polyphosphat-4-phosphatase type II gene (INPP4b gene), was shown to be significantly associated with MS.

TABLE 3

| Haplotype | pHaploScore | snp1 | snp2 | snp3 |
|---|---|---|---|---|
| 1 | 8.92E−04 | rs17717651 | rs2059510 | rs13102150 |
| 2 | 1.93E−03 | rs2059510 | rs13102150 | rs16998560 |
| 3 | 1.08E−02 | rs2667096 | rs2667101 | rs168059 |
| 4 | 1.49E−02 | rs13107432 | rs17717651 | rs2059510 |
| 5 | 2.02E−02 | rs16998560 | rs4975311 | rs2874870 |
| 6 | 2.74E−02 | rs336307 | rs168061 | rs2636637 |

Accordingly one preferred form of embodiment of the invention is characterised in that a haplotype comprising single nucleotide polymorphisms (SNPs) of the nucleobases at base position 143470133 (rs13102150), base position 143459907 (rs2059510) and base position 143453079 (rs17717651) of human chromosome 4 in the inositol polyphosphat-4-phosphatase type II gene (INPP4b gene) is used for the diagnosis or pre-diagnosis of multiple sclerosis or for determining the risk of developing multiple sclerosis. Particularly preferably this haplotype is characterised in that the polymorphisms cover a replacement of the base cytosine with adenine at base position 143470133 (rs13102150), a replacement of the base thymine with cytosine at base position 143459907 (rs2059510) and a replacement of the base cytosine with adenine at base position 143453079 (rs17717651).

In other words in the case of a haplotype in which at base position 143470133 another base, particularly adenine, is present in place of cytosine, at base position 143459907 another base, particularly cytosine, is present in place of thymine and at base position 143453079 another base, particularly adenine is present in place of cytosine the patient is diagnosed or pre-diagnosed with MS or the patient is classified as being at increased risk of developing the disease.

In the method in accordance with the invention for diagnosing or pre-diagnosing MS or determining the risk of a study participant developing MS, at least the base at base position 143470133 (rs13102150) of human chromosome 4 in the inositol polyphosphate-4-phosphatase type II gene (INPP4b gene) is analysed, whereby if a base other than cytosine, more particularly an adenine, is present there instead of a cytosine, the proband is diagnosed with multiple sclerosis or the proband is classified as being at increased risk of developing the disease.

In a preferred form of embodiment of the method of diagnosing or pre-diagnosing multiple sclerosis or determining the risk of a proband of developing multiple sclerosis, at least the bases at base position 143470133 (rs13102150), base position 143459907 (rs2059510) and base position 143453079 (rs17717651) of human chromosome 4 in the inositol polyphosphate-4-phosphatase type II gene (INPP4b gene) are analysed, whereby if at base position 143470133 a base other than cytosine, particularly an adenine, is present in place of a cytosine and at base position143459907 a base other than thymine, particularly a cytosine, is present in place of a thymine and at base position 143453079 a base other than cytosine, particularly an adenine, is present in place of a cytosine, the proband is diagnosed with multiple sclerosis or the proband is classified as being at increased risk of developing the disease.

In the method in accordance with the invention, bodily material, preferable cell and/or tissue material, is taken from the proband. Particularly preferably blood samples are taken. From this DNA as the carrier of the genetic information is isolated and the sequence is then identified and compared with the reference sequence at the corresponding point of human chromosome 4 and the Inpp4b gene, respectively. There are many methods suitable and known to a person skilled in the art for identifying the sequence, which also include sequencing of the DNA. For methods requiring DNA replication the amplification of at least a part of the gene can be carried out with methods known to a person skilled in the art. Examples which can be mentioned here are PCR and/or LCR. Alternatively there are methods such as “self-sustained sequence replication”, transcriptional amplification systems or Q-beta replicase.

As sequencing is very laborious, methods are preferably used for identification which do not require full sequencing. For this, methods such as pyrosequencing methods, which are, for example, provided by the company QIAGEN, specific methods of detecting DNA differences such as the Taqman® PCR (Real-Time PCR-Based Assays), offered for example by the company AB applied biosystems, or electrochemical approaches to DNA detection, such as GENSORIC® by the company Gensoric GmbH can be cited. Other methods used for identification are described in EP 1 388 589 A1 (paragraphs [0111] ff.).

### DESCRIPTION OF THE FIGURES

FIG. 1 Shows two of the applied fine mapping strategies. In part A of the figure an F1 backcross with one parenteral strain (FO) is shown. In part B the F1 inter-crossing with a sibling is shown (F1).

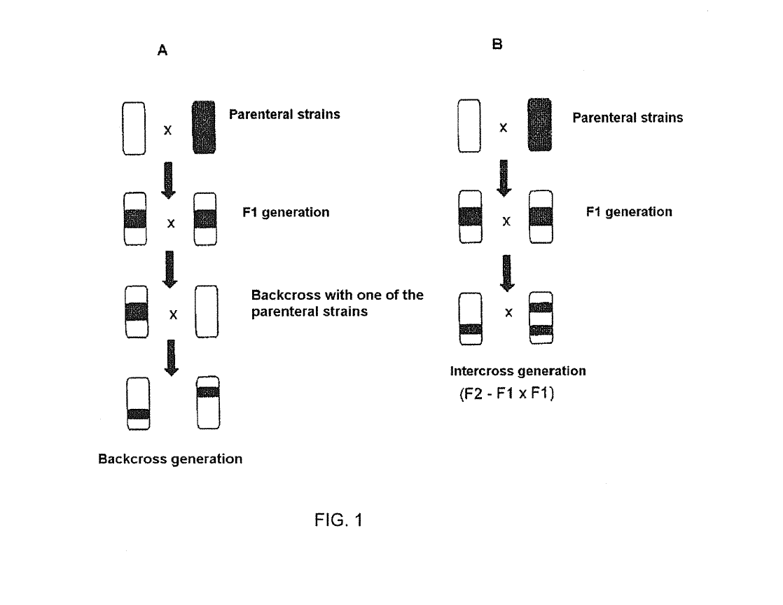

FIG. 2 Shows the comparison of chromosomal fragments of human, mouse and rat. Shown is the location of the QTL EAE 31 in all three species.

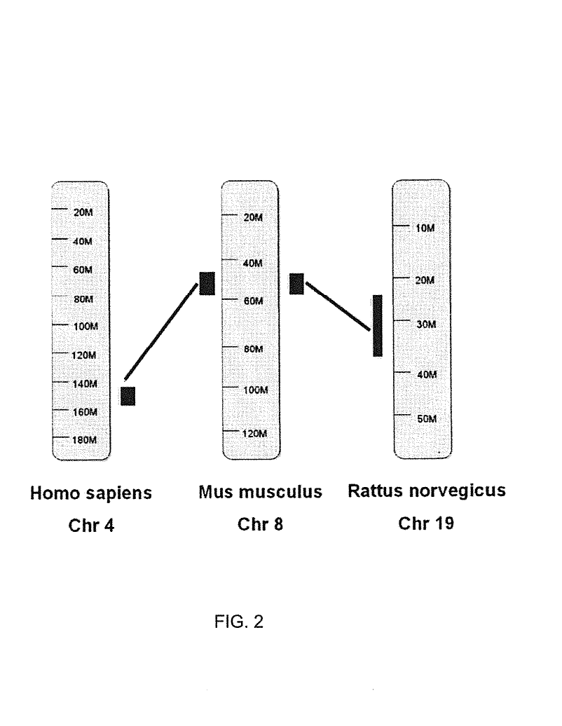

FIG. 3 Shown schematically is the EAE 31 QTL in human (A) and in mouse (B). The fine mapping of the EAE 31 points to the gene Inpp4b.

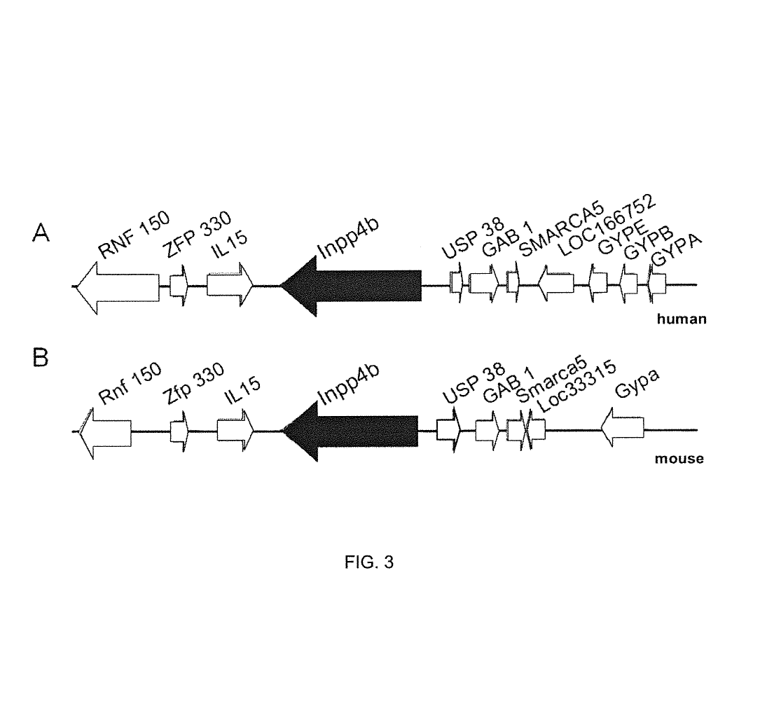

FIG. 4 Shows the haplotype analysis, indicating the global p-values for sub-haplotypes based on table 3. The line between 2.5 and 3.0 shows the significance limit value after correction for multiple testing.

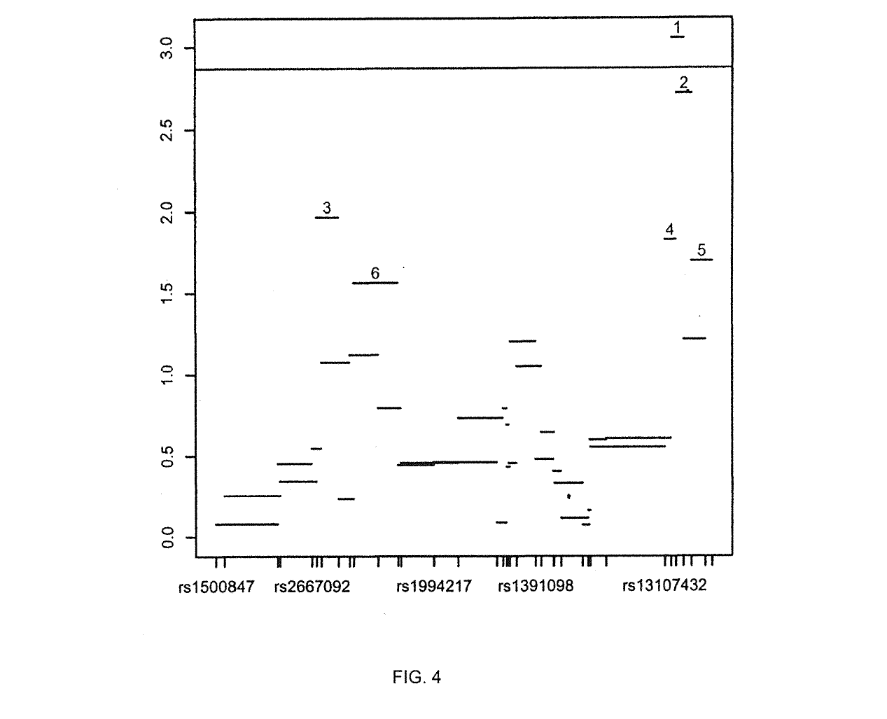

1-4. (canceled)

5. A method of diagnosing or pre-diagnosing MS or determining the risk of a proband developing MS characterised in that at least the base at base position 143470133 of human chromosome 4 in the inositol polyphosphate-4-phosphatase type II gene is analysed, whereby if another base is present there in place of a cytosine, the proband is diagnosed with multiple sclerosis or the proband is classified as being at increased risk of developing the disease.

6. The method of diagnosing or pre-diagnosing MS or determining the risk of a proband of developing MS in accordance with claim 5 characterised in that if at base position 143470133 (rs13102150) of human chromosome 4 in the inositol polyphosphate-4-phosphatase type II gene an adenine is present instead of a cytosine the proband is diagnosed with multiple sclerosis or the proband is classified as being at increased risk of developing the disease.

7. The method of diagnosing or pre-diagnosing multiple sclerosis or determining the risk of a proband developing multiple sclerosis characterised in that at least the bases at base position 143470133, base position 143459907 and base position 143453079 of human chromosome 4 in the inositol polyphosphate-4-phosphatase type II gene (INPP4b gene) are analysed whereby if at base position 143470133 another base is present in place of a cytosine and
at base position 143459907 another base is present in place of a thymine and
at base position 143453079 another base is present in place of a cytosine the proband is diagnosed with multiple sclerosis or the proband is classified as being at increased risk of developing the disease.

8. The method of diagnosing or pre-diagnosing multiple sclerosis or determining the risk of a proband of developing multiple sclerosis in accordance with claim 7 characterised in that if
at base position 143470133 an adenine is present in place of a cytosine and
at base position 143459907 a cytosine is present in place of a thymine and
at base position 143453079 an adenine is present in place of a cytosine, the proband is diagnosed with multiple sclerosis or the proband is classified as being at increased risk of developing the disease.

9. The method in accordance with claim 5 or 8 characterised in that bodily material is taken from the proband.

10. The method in accordance with claim 9 characterised in that blood samples are taken from the proband.

11. The method in accordance with claim 9 characterised in that the DNA to be analysed is isolated from the bodily material, and the sequence is then identified.

12. The method in accordance with claim 9 characterised in that bodily material comprises cell and/or tissue material.

13. The method in accordance with claim 11 characterised in that the bodily material comprises cell and/or tissue material.

14. The method in accordance with claim 13 wherein said cell and/or tissue material comprises blood samples.
